(19) United States
(12) Patent Application Publication
(10) Pub. No.: US 2024/0206800 A1
(43) Pub. Date: Jun. 27, 2024

(54) SYSTEMS AND METHODS FOR CONCUSSION DETECTION AND REPORTING

(71) Applicants: Armon Mighani, Marietta, GA (US); Patrick Mauricio Astorga, Marietta, GA (US); Tate Jackson Smith, Marietta, GA (US)

(72) Inventors: Armon Mighani, Marietta, GA (US); Patrick Mauricio Astorga, Marietta, GA (US); Tate Jackson Smith, Marietta, GA (US)

(21) Appl. No.: 18/392,283

(22) Filed: Dec. 21, 2023

Related U.S. Application Data
(60) Provisional application No. 63/434,792, filed on Dec. 22, 2022.

(51) Int. Cl. A61B 5/00; A42B 3/04; G16H 20/00

(52) U.S. Cl. CPC A61B 5/4064; A42B 3/046; A61B 5/0022; A61B 5/6803; A61B 5/746; A61B 5/7465; G16H 20/00; A61B 2503/10; A63B 2220/53

(57) ABSTRACT

Disclosed systems and methods relate to concussion detection and reporting. In an example, a method involves receiving sensor data from an accelerometer. The method further involves determining an impact acceleration from the sensor data and comparing the impact acceleration to a threshold value. The threshold value may be adjustable based on a user's age. When the impact acceleration is less than the threshold value, the method involves transmitting the sensor data to a server. When the impact acceleration equals or exceeds the threshold value, the method involves transmitting an alert and transmitting the sensor data to a server.

### CROSS-REFERENCE TO RELATED APPLICATION

The present patent application claims of the benefit of U.S. Patent Application No. 63/434,792 titled “TECHNIQUES FOR CONCUSSION DETECTION AND REPORTING” filed Dec. 22, 2022, the entire contents of which are incorporated by reference herein for all purposes.

### FIELD OF INVENTION

The present invention relates generally to wearable smart devices, data processing, and concussion detection and more particularly to systems and methods for using accelerometer data collected by a concussion detecting circuit to detect and report concussions.

### BACKGROUND

Concussions can cause severe damage to an injured person's mind. Undiagnosed concussions are particularly harmful, because subsequent impacts have a much higher probability of leading to permanent and severe brain damage for those who recently suffered a concussion. Further, undiagnosed and unreported concussions may go untreated, worsen over time, and leave athletes vulnerable to receive additional concussions. In fact, roughly 39% of additional impacts after a concussion lead to permanent and severe brain damage. This statistic is particularly alarming, because roughly 1 out of every 3 student athletes suffer a concussion without even realizing it. Further, roughly half of all concussions go unreported by athletes for other reasons, such as the athletes not wanting to be removed from play. Thus, there exists a need for systems and methods to detect concussions and report the concussions to coaches and athletes.

### SUMMARY

In an aspect, a method for concussion detection and reporting involves receiving sensor data. The sensor data may include acceleration data measured by an accelerometer. The method further involves determining impact acceleration from the sensor data and comparing the impact acceleration to predetermined threshold values. The predetermined threshold values may vary based on a user's age or physical stature such as height and weight. The method further involves determining whether a concussion is detected based on the comparison and when a concussion is detected, transmitting an alert, and transmitting the sensor data to a server. In some cases, the method further involves, when a concussion is not detected, transmitting the sensor data to the server.

According to other embodiments, a system for concussion detection and reporting may include a concussion detecting circuit with a processing unit, sensor, and communication module. The concussion detecting circuit receives sensor data from the sensor, such as an accelerometer. The processing unit may compare the sensor data to predetermined threshold values to determine whether a concussion is detected. Based on the determination, the concussion detecting circuit generates and transmits an alert of the concussion to a user and transmits the sensor data to a server.

The above methods can be implemented as computer-executable program instructions stored in a tangible computer-readable media and/or operating within a computer processor or other processing device and attached memory.

### BRIEF DESCRIPTION OF THE DRAWINGS

A full and enabling disclosure is set forth more particularly in the remainder of the specification. The specification makes reference to the following appended figures.

FIG. 1 shows an illustrative example of a concussion detecting circuit.

FIG. 2 shows an illustrative example of a concussion detecting circuit and reporting system.

FIG. 3 illustrates a flow chart for a method of detecting and reporting concussions.

FIG. 4 shows an illustrative example of a concussion detecting circuit.

FIGS. 5A-5C show illustrative examples of the concussion detecting circuit integrated into wearable sports gear.

### DETAILED DESCRIPTION

Reference will now be made in detail to various and alternative illustrative examples and to the accompanying drawings. Each example is provided by way of explanation, and not as a limitation. It will be apparent to those skilled in the art that modifications and variations can be made. For instance, features illustrated or described as part of one example may be used on another example to yield a still further example. Thus, it is intended that this disclosure include modifications and variations as come within the scope of the appended claims and their equivalents.

### Exemplary Concussion Detecting Circuit

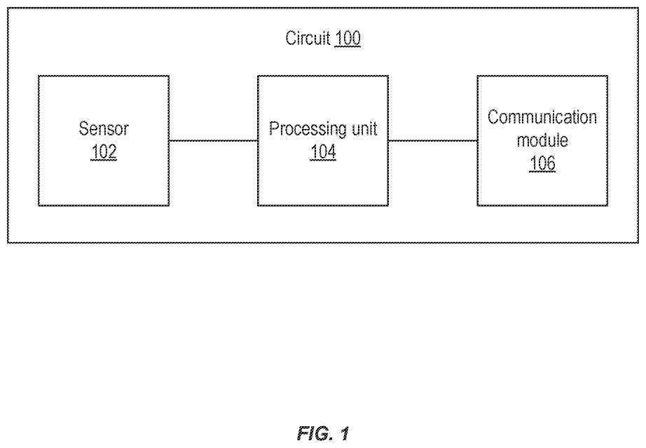

FIG. 1 shows an illustrative example of a concussion detecting circuit 100. The concussion detecting circuit 100 comprises a sensor 102, a processing unit 104, and a communication module 106. FIG. 1 shows the processing unit 104 connected to the sensor 102 and communication module 106. In other examples, the communication module 106 and sensor 102 may also be connected. The concussion detecting circuit 100 may include additional components not shown, including various switches, tabs, buttons, LEDs, and power sources such as a battery. During operation, the concussion detecting circuit 100 is located at an area adjacent to a user's head or neck. For example, the concussion detecting circuit 100 may be located in or on wearable clothing or sports gear worn at an area adjacent to the user's head, such as in a helmet, a headband, a hat, glasses, goggles, or a mouthpiece.

The sensor 102 generates sensor data representing impact acceleration of the concussion detecting circuit 100. In some examples, the sensor is a high G accelerometer. In other examples, the sensor may be a pressure sensor or an impact sensor, such as a piezoelectric sensor or a piezoresistive sensor.

The processing unit 104 may include various processors to carry out program instructions, as well as memory to store program instructions and sensor data. In some examples, the processing unit 104 is a microcontroller. The processing unit 104 receives sensor data from the sensor 102, determines an impact acceleration from the sensor data, and based on the impact acceleration, determines whether the sensor data indicates a concussion was sustained by the user. The processing unit 104 may store the determination in memory and transmit the determination to the communication module 106.

In one example, the processing unit 104 detects concussions by comparing the sensor data to predetermined threshold values. Predetermined threshold values may include threshold impact acceleration measurements associated with concussions. For example, concussions generally occur when a user's head is hit by a force between 70 Gs and 120 Gs (Gs meaning G-forces).

In one example, the predetermined threshold values may represent different levels of severity of the impact or likelihood of sustained concussion. For example, a first threshold value may be 70 Gs, indicating that a user suffered a minor concussion. A second threshold measurement may be 80 Gs, indicating that a user suffered a moderate concussion, and a third threshold measurement may be 90 Gs, indicating that a user suffered a severe concussion. The processing unit 104 may assign a grade of concussion based on which threshold measurements the impact acceleration exceeds, and generate an alert based on the grade of concussion. For example, the processing unit 104 may determine that when the impact acceleration is greater than 90 Gs, a user likely suffered a severe concussion. The processing unit 104 may generate an alert that recommends the user immediately be removed from a game and seek immediate medical attention. The processing unit 104 may adjust the recommendation based on the impact acceleration. For example, when the processing unit 104 determines the impact acceleration to be 70 Gs, an alert may include a recommendation that a user be tested for a concussion before potentially returning to the game.

The communication module 106 may transmit sensor data from the sensor 102 and concussion determinations from the processing unit 104 to an external device (not shown). The communication module 106 may use various wireless technologies including: WiFi, Bluetooth, RF, 3g, 4g, and 5g. In some examples, the communication module 106 may transmit sensor data and concussion determinations to an external device such as a smartphone, laptop, or tablet. In other examples, the communication module 106 may transmit sensor data and concussion determinations to a server.

In further examples, the communication module 106 may transmit alerts generated by the processing unit 104 to a server or an external device such as a smartphone, laptop, or tablet. For example, when the processing unit 104 determines that a concussion was likely suffered by a user, the communications module 106 may transmit an alert to a smartphone application or to a smartphone using an SMS. The alert may include the impact acceleration determined by the processing unit 104 and a recommendation such as a recommendation to remove a user from a game and seek immediate medical attention.

### Illustrative Example of Communication Between the Concussion Detecting Circuit and Concussion Tracking Server

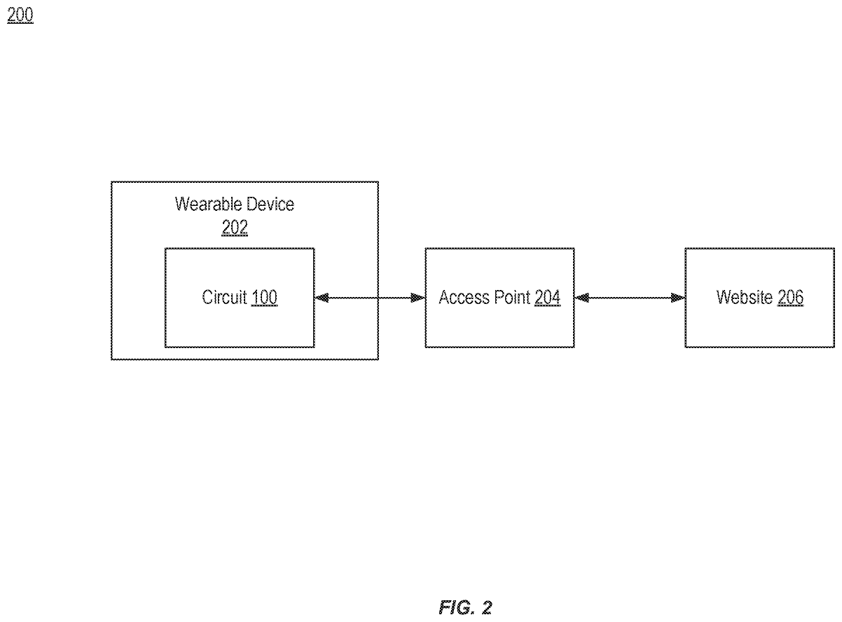

FIG. 2 shows an illustrative example of the concussion detecting circuit 100 from FIG. 1 communicating with a concussion tracking server 206. The concussion detecting circuit 100 is located in or on wearable device 202. The concussion detecting circuit 100 communicates to an access point 204. The access point 204 establishes a local wireless network connecting the concussion detecting circuit 100 to the internet or a local network. In one example, the access point 204 may be a WiFi access point.

When connected to the local network or internet, the concussion detecting circuit 100 may transmit sensor data and concussion determinations to the concussion tracking server 206 through access point 204. The concussion tracking server 206 logs concussion determinations and graphs sensor data.

In further examples, the concussion tracking server 206 also receives alerts from the concussion detecting circuit 100 including the recommendation and stores the alerts in a log associated with a user, groups of users, or an entity. For example, the log may include information associated with groups of users such as a team, an entity such as the school associated with the team, or past alerts associated with an individual user.

In some examples, the concussion tracking server 206 may include a user profile. The user profile may include information associated with a user such as the team the user plays for, the sport the user plays, the user's position on his or her team, the user's age, and the user's height and weight.

In some examples, the concussion tracking server 206 may allow for filtering and arranging of information associated with individuals, teams, and entities to identify which sport positions (e.g., linebacker, wide receiver, quarterback) of teams have higher likelihoods of suffering from concussions and identify and report which teams suffer from higher incidences of concussions. In some examples, the concussion tracking server 206 may provide reports to users, such as coaches and athletes, regarding a team's or user's history of concussions, such as time and date of concussion and number of concussions suffered by a team or user.

The concussion tracking server 206 may provide users, such as coaches, with information indicating an increase in concussions for his or her team over time, which the user may use to determine whether to include an athlete in an upcoming game or to determine whether the team should be further trained to avoid concussions (e.g., such as providing tackling training in football, heading training in soccer).

### Illustrative Example of Concussion Detection and Reporting

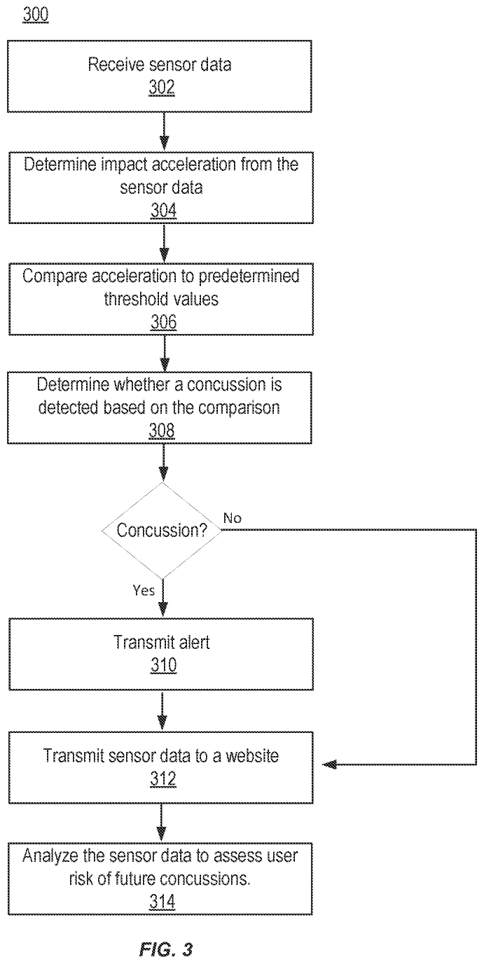

FIG. 3 illustrates a flow chart of method 300 for detecting and reporting concussions. At block 302, method 300 includes a concussion detecting circuit receiving sensor data. The sensor data may represent impact acceleration exerted onto a sensor located at an area adjacent to a user's head.

At block 304, method 300 includes the concussion detecting circuit determining the impact acceleration from the sensor data. The concussion detecting circuit may calculate the impact acceleration from the sensor data, such as by translating the sensor data into Gs using a processing unit. For example, the concussion detecting circuit may measure the x-axis, y-axis, and z-axis movement, force, or force per unit mass (e.g., Gs) from the concussion detecting circuit as scalars or vectors. The concussion detecting circuit may then calculate a three-dimensional vector sum of these x-, y-, and z-axis vectors.

At block 306, method 300 includes the processing unit comparing acceleration to predetermined threshold values and at block 308, the processing unit determines whether a concussion is detected based on the comparison at block 306. The predetermined threshold values may be impact acceleration values indicating a concussion has been sustained. For example, the predetermined threshold values may be values indicating 70 Gs of force. In other examples, the predetermined threshold values may vary based on information associated with the user. For example, if the user is an adolescent, the predetermined threshold values may be 50 Gs or 60 Gs instead of 70 Gs.

When the concussion detecting circuit determines a concussion has occurred, method 300 proceeds to block 310 and the concussion detecting circuit transmits an alert. The concussion detecting circuit may transmit the alert to an external device such as a smartphone, laptop, or tablet. The alert may include information indicating that a user has suffered a concussion and information associated with the severity of the concussion based on the impact acceleration.

At block 312, the concussion detecting circuit transmits the sensor data to a server. The server logs the sensor data and concussion determinations and graphs the sensor data. The user may view the concussion determinations and graphs on the server. In some examples, the concussion detecting circuit may store the sensor data in local memory. In further examples, a user may download the sensor data from the local memory and upload the sensor data to a server. In further examples, the concussion detecting circuit may communicate with the server using WebSocket. In further examples, the concussion detecting circuit uses other communication protocols such as NFS, NIS+, and DNS. In some examples, the website stores the sensor data for individual users in a user profile.

At block 314, the concussion detecting circuit analyzes the sensor data to assess risk of future concussions. For example, when a user suffers a concussion, the user may become more susceptible to future concussions. The concussion detecting circuit may lower predetermined threshold values for determining whether the user suffered a concussion based on past concussions suffered by the user. In some examples, the concussion detecting circuit analyzes the sensor data using an artificial intelligence or machine learning model. In further examples, analysis of the sensor data to assess risk of future concussions may occur using a separate computing device or cloud computing device using the sensor data transmitted to the website. For example, a machine learning model may use the sensor data of individual users to predict the likelihood the user will receive a concussion, the susceptibility of the user in receiving a concussion, and suggest an updated predetermined threshold value of impact acceleration for the concussion detecting circuit to use in determining whether the user likely received a concussion.

When a concussion is not detected by the concussion detecting circuit, method 300 may also proceed to block 312.

### Illustrative Example of a Concussion Detection Circuit

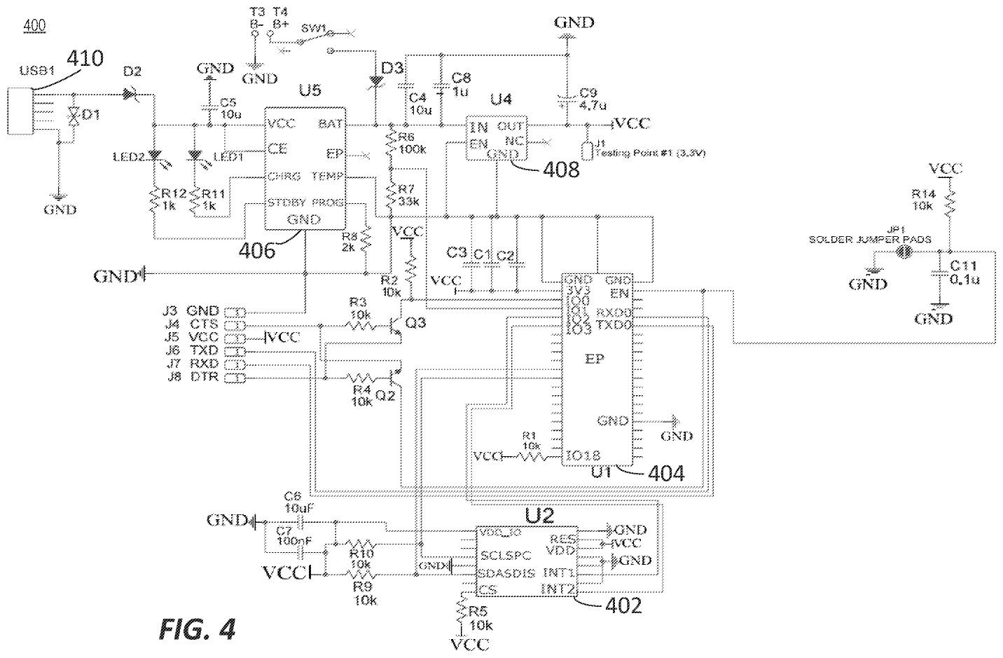

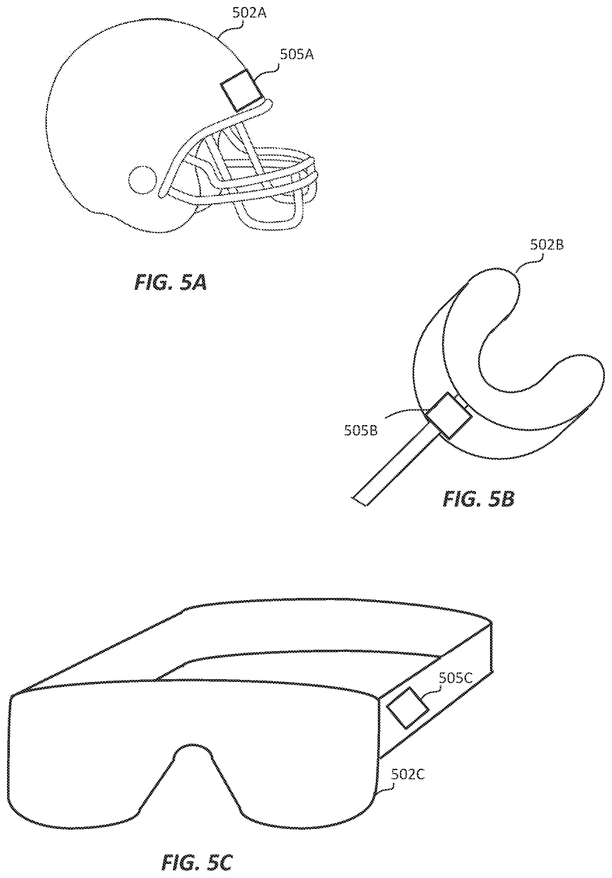

FIG. 4 provides an additional illustrative example of a concussion detecting circuit 400 including an accelerometer 402, a joint communications module and processing unit 404, a battery charging module 406, a voltage regulator 408, and a USB connector 410. The concussion detecting circuit 400 may be located in or on a variety of sports gear, such as sports gear worn adjacent to or connected to a wearer's head. Examples of the concussion detecting circuit integrated on or in sports gear are further described in FIGS. 5A-5C.

FIG. 4 shows the accelerometer 402 connected to an integrated circuit including the joint communications module and processing unit 404. In some examples, the concussion detecting circuit 400 may include separate components for the communications module and processing unit, as shown and further described in FIG. 1.

The accelerometer 402 measures acceleration of the concussion detecting circuit 400 and provides sensor data to the joint communications module and processing unit 404. The joint communications module and processing unit 404 includes onboard memory to store program instructions and sensor data. Further, the joint communications module and processing unit 404 determines an impact acceleration from the sensor data and determines whether the impact acceleration indicates a user likely sustained a concussion. Additional description of determining whether a user suffered a concussion using the concussion detecting circuit 400 is provided in the description of FIG. 1.

The joint communications and processing unit 404 may transmit the impact acceleration, sensor data, and determination to a server or external device, such as a smartphone, laptop, or tablet.

The battery charging module 406 connects to the USB connector 410, a battery (not shown), and a voltage regulator 408. An external power source may connect to the concussion detecting circuit 400 through the USB connector 410 and the battery charging module 406 may use constant-current/constant-voltage (CC/CV) charging methods to charge the rechargeable battery and protect against overcharge of the battery. The voltage regulator 408 enables operation of the joint communications and processing unit 404 by providing a constant voltage.

### Illustrative Examples of Wearable Sports Gear

FIGS. 5A-5C provide examples of pieces of protective gear including a concussion detecting circuit integrated in or on the piece of protective gear. The pieces of protective gear may include various examples of wearable sports gear. For example, FIG. 5A illustrates a football helmet 502A with a concussion detecting circuit 505A integrated in the helmet. In some examples, the concussion detecting circuit 505A is attached inside protective pads within the football helmet 502A. In further examples, the concussion detecting circuit 505A is attached to an exterior surface of the football helmet 502A.

FIG. 5B illustrates a mouthpiece 502B with a concussion detecting circuit 505B integrated within the mouthpiece 502B. In some examples, the concussion detecting circuit 505B is attached to a strap protruding from the mouthpiece 502B. In further examples, the concussion detecting circuit 505B is attached within the mouthpiece 502B.

FIG. 5C illustrates goggles 502C with a concussion detecting circuit 505C integrated within the goggles 502C. In some examples, the concussion detecting circuit 505C is attached to an interior or exterior surface of the goggles 502C such as in or on a strap of the goggles.

In the foregoing specification, aspects of the invention are described with reference to specific aspects thereof, but those skilled in the art will recognize that the invention is not limited thereto. Various features and aspects of the above-described invention may be used individually or jointly. Further, aspects can be utilized in any number of environments and applications beyond those described herein without departing from the broader spirit and scope of the specification. The specification and drawings are, accordingly, to be regarded as illustrative rather than restrictive.

Further, while the present subject matter has been described in detail with respect to specific aspects thereof, it will be appreciated that those skilled in the art, upon attaining an understanding of the foregoing, may readily produce alterations to, variations of, and equivalents to such aspects. Accordingly, it should be understood that the present disclosure has been presented for purposes of example rather than limitation and does not preclude inclusion of such modifications, variations, and/or additions to the present subject matter as would be readily apparent to one of ordinary skill in the art.

### EXAMPLES

A collection of exemplary embodiments, including at least some explicitly enumerated as “Examples” providing additional description of a variety of example types in accordance with the concepts described herein are provided below. These examples are not meant to be mutually exclusive, exhaustive, or restrictive; and the invention is not limited to these examples but rather encompasses all possible modifications and variations within the scope of the issued claims and their equivalents.

Example 1: A method comprising: receiving sensor data from an accelerometer; determining impact acceleration from the sensor data; comparing the impact acceleration to a threshold value, wherein when the impact acceleration equals or exceeds the threshold value: transmit an alert; transmit the sensor data to a server; and wherein when the impact acceleration is less than the threshold value: transmit the sensor data to the server.

Example 2: The method of any of the preceding or subsequent examples or combination of examples, wherein the threshold value is 70 Gs of force.

Example 3: The method of any of the preceding or subsequent examples or combination of examples, wherein the threshold value is adjustable based on age of a user.

Example 4: The method of any of the preceding or subsequent examples or combination of examples, wherein the alert is transmitted using one of Network File System (NFS), NIS+, and DNS protocols.

Example 5: The method of any of the preceding or subsequent examples or combination of examples, wherein the accelerometer is connected to one or more of: a helmet, a headband, a hat, glasses, goggles, and a mouthpiece.

Example 6: The method of any of the preceding or subsequent examples or combination of examples, wherein the alert and sensor data are transmitted over one or more of: WiFi, Bluetooth, 3g, 4g, and 5g.

Example 7: The method of any of the preceding or subsequent examples or combination of examples, wherein the alert includes a recommendation to remove a player from a game.

Example 8: A system comprising: an accelerometer; a communications module configured to communicate with a server; and a non-transitory computer-readable medium storing computer-executable program instructions; and a processing device communicatively coupled to the non-transitory computer-readable medium for executing the computer-executable program instructions, wherein executing the computer-executable program instructions configures the processing device to perform operations comprising: receiving sensor data from the accelerometer; determining impact acceleration from the sensor data; comparing the impact acceleration to a threshold value, wherein when the impact acceleration equals or exceeds the threshold value: transmit an alert; transmit the sensor data to a server; and wherein when the impact acceleration is less than the threshold value: transmit the sensor data to the server.

Example 9: The system of any of the preceding or subsequent examples or combination of examples, wherein the threshold value is 70 Gs of force.

Example 10: The system of any of the preceding or subsequent examples or combination of examples, wherein the threshold value is adjustable based on age of a user.

Example 11: The system of any of the preceding or subsequent examples or combination of examples, wherein the alert is transmitted using one of Network File System (NFS), NIS+, and DNS protocols.

Example 12: The system of any of the preceding or subsequent examples or combination of examples, wherein the accelerometer is located in one or more of: a helmet, a headband, a hat, glasses, goggles, and a mouthpiece.

Example 13: The system of any of the preceding or subsequent examples or combination of examples, wherein the alert and sensor data are transmitted over one or more of: WiFi, Bluetooth, 3g, 4g, and 5g.

Example 14: The system of any of the preceding or subsequent examples or combination of examples, wherein the alert includes a recommendation to remove a player from a game.

Example 15: A non-transitory computer-readable storage medium storing computer-executable program instructions, wherein when executed by a processing device, the computer-executable program instructions cause the processing device to perform operations comprising: receiving sensor data from an accelerometer; determining impact acceleration from the sensor data; comparing the impact acceleration to a threshold value, wherein when the impact acceleration equals or exceeds the threshold value: transmit an alert; transmit the sensor data to a server; and wherein when the impact acceleration is less than the threshold value: transmit the sensor data to the server.

Example 16: The non-transitory computer readable medium of any of the preceding or subsequent examples or combination of examples, wherein the threshold value is 70 Gs of force.

Example 17: The non-transitory computer readable medium of any of the preceding or subsequent examples or combination of examples, wherein the threshold value is adjustable based on age of a user.

Example 18: The non-transitory computer readable medium of any of the preceding or subsequent examples or combination of examples, wherein the alert is transmitted using one of Network File System (NFS), NIS+, and DNS protocols.

Example 19: The non-transitory computer readable medium of any of the preceding or subsequent examples or combination of examples, wherein the accelerometer is located in one or more of: a helmet, a headband, a hat, glasses, goggles, and a mouthpiece.

Example 20: The non-transitory computer readable medium of any of the preceding or subsequent examples or combination of examples, wherein the alert and sensor data are transmitted over one or more of: WiFi, Bluetooth, 3g, 4g, and 5g.

Different arrangements of the components depicted in the drawings or described above, as well as components and steps not shown or described are possible. Similarly, some features and sub-combinations are useful and may be employed without reference to other features and sub-combinations. Embodiments of the invention have been described for illustrative and not restrictive purposes, and alternative embodiments will become apparent to readers of this patent. Accordingly, the present invention is not limited to the embodiments described above or depicted in the drawings, and various embodiments and modifications may be made without departing from the scope of the claims below.

What is claimed is:

1. A method comprising:
receiving sensor data from a high G accelerometer within a piece of protective gear;
determining impact acceleration from the sensor data;
comparing the impact acceleration to a threshold value, wherein when the impact acceleration equals or exceeds the threshold value:
transmit an alert;
transmit the sensor data to a server; and
wherein when the impact acceleration is less than the threshold value:
transmit the sensor data to the server.

2. The method of claim 1, wherein the threshold value is 70 Gs of force.

3. The method of claim 1, wherein the threshold value is adjustable based on age of a user.

4. The method of claim 1, wherein the alert is transmitted using one of Network File System (NFS), NIS+, and DNS protocols.

5. The method of claim 1, wherein the piece of protective gear is one or more of: a helmet, a headband, a hat, glasses, goggles, and a mouthpiece.

6. The method of claim 1, wherein the alert and sensor data are transmitted over one or more of: WiFi, Bluetooth, 3g, 4g, and 5g.

7. The method of claim 1, wherein the alert includes a recommendation to remove a player from a game.

8. A system comprising:
a high G accelerometer within a piece of protective gear;
a communications module configured to communicate with a server; and
a non-transitory computer-readable medium storing computer-executable program instructions; and
a processing device communicatively coupled to the non-transitory computer-readable medium for executing the computer-executable program instructions, wherein executing the computer-executable program instructions configures the processing device to perform operations comprising:
receiving sensor data from the high G accelerometer;
determining impact acceleration from the sensor data;
comparing the impact acceleration to a threshold value, wherein when the impact acceleration equals or exceeds the threshold value:
transmit an alert;
transmit the sensor data to a server; and
wherein when the impact acceleration is less than the threshold value:
transmit the sensor data to the server.

9. The system of claim 8, wherein the threshold value is 70 Gs of force.

10. The system of claim 8, wherein the threshold value is adjustable based on age of a user.

11. The system of claim 8, wherein the alert is transmitted using one of Network File System (NFS), NIS+, and DNS protocols.

12. The system of claim 8, wherein the piece of protective gear is one or more of: a helmet, a headband, a hat, glasses, goggles, and a mouthpiece.

13. The system of claim 8, wherein the alert and sensor data are transmitted over one or more of: WiFi, Bluetooth, 3g, 4g, and 5g.

14. The system of claim 8, wherein the alert includes a recommendation to remove a player from a game.

15. A non-transitory computer-readable storage medium storing computer-executable program instructions, wherein when executed by a processing device, the computer-executable program instructions cause the processing device to perform operations comprising:
receiving sensor data from a high G accelerometer within a piece of protective gear;
determining impact acceleration from the sensor data;
comparing the impact acceleration to a threshold value, wherein when the impact acceleration equals or exceeds the threshold value:
transmit an alert;
transmit the sensor data to a server; and
wherein when the impact acceleration is less than the threshold value:
transmit the sensor data to the server.

16. The non-transitory computer-readable medium of claim 15, wherein the threshold value is 70 Gs of force.

17. The non-transitory computer-readable medium of claim 15, wherein the threshold value is adjustable based on age of a user.

18. The non-transitory computer-readable medium of claim 15, wherein the alert is transmitted using one of Network File System (NFS), NIS+, and DNS protocols.

19. The non-transitory computer-readable medium of claim 15, wherein the piece of protective gear is one or more of: a helmet, a headband, a hat, glasses, goggles, and a mouthpiece.

20. The non-transitory computer-readable medium of claim 15, wherein the alert and sensor data are transmitted over one or more of WiFi, Bluetooth, 3g, 4g, and 5g.
